(19) United States
(12) Patent Application Publication
(10) Pub. No.: US 2023/0169696 A1
(43) Pub. Date: Jun. 1, 2023

(54) Transfer of Alignment Accuracy Between Visible Markers Used with Augmented Reality Displays

(71) Applicant: Novarad Corporation, American Fork, UT (US)

(72) Inventors: Wendell Arlen Gibby, Mapleton, UT (US); Steven Todd Cvetko, Draper, UT (US)

(21) Appl. No.: 17/536,011

(22) Filed: Nov. 27, 2021

(51) Int. Cl. G06T 11/00; G06V 20/20; G16H 30/40

(52) U.S. Cl. CPC G06T 11/00; G06V 20/20; G16H 30/40; G06T 2200/24; G06T 2210/41; G06K 7/1417

(57) ABSTRACT

A technology is described for using an augmented reality (AR) headset to co-localize an image data set with a body of a person. A method may include registering one or more initial visible markers attached to the body of the person, and the optical code may be located in a fixed position relative to the image visible marker. The coordinate system of the one or more initial visible markers may be transferred to the one or more additional visible markers. The use of the one or more additional visible markers may be emphasized to align of the image data set with the body of the person.

### BACKGROUND

Mixed or augmented reality is an area of computing technology where views from the physical world and images from virtual computing worlds may be combined into a mixed reality world. In mixed reality, people, places, and objects from the physical world and virtual worlds become a blended visual and audio environment. A mixed reality experience may be provided through existing commercial or custom software along with the use of VR (virtual reality) or AR (augmented reality) headsets.

Augmented reality (AR) is an example of mixed reality where a live direct view (or an indirect view) of a physical, real-world environment is augmented or supplemented by computer-generated sensory input such as sound, video, graphics or other data. Augmentation is performed as a real-world location is viewed and in context with environmental elements. With the help of advanced AR technology (e.g. adding computer vision and object recognition) the information about the surrounding real world of the user becomes interactive and may be digitally modified.

An issue faced by AR systems or AR headsets is identifying a position and orientation of an object in the physical world with a high degree of precision. Similarly aligning the position of a virtual element with a live view of a real-world environment and objects may be challenging. The alignment resolution of an AR headset may be able to align a virtual object to a physical object being viewed but the alignment resolution may only be aligned to within a few centimeters. Providing alignment of a virtual object to a physical object to within a few centimeters may be useful for entertainment, games and less demanding applications but greater positioning and alignment resolution for AR systems may be desired in the scientific, engineering and medical disciplines. As a result, positioning and alignment processes may be done manually which can be time consuming, cumbersome, and inaccurate.

### BRIEF DESCRIPTION OF THE DRAWINGS

FIG. 1 illustrates an example augmented reality (AR) environment in which an image data set of a patient may be aligned to actual views of the patient using one or more visible markers with image visible markers attached to the patient;

FIG. 2 illustrates an example of an visible marker with an image visible marker that is affixed to a patient;

FIG. 3 illustrates an example of initial visible markers that may be affixed to a body of a patient near a location where an incision may be made.

FIG. 4 illustrates an example of a view of an open incision that has caused some of the initial visible markers (as in FIG. 3) to move but the additional visible markers have not moved.

FIG. 5 illustrates a view of an example of initial visible markers that may have been partially covered by an object or material.

FIG. 6 is a flowchart of an example method of changing alignment from initial visible markers and image visible markers to additional visible markers and image visible markers.

FIG. 7 illustrates an example system that may be employed in using one or more visible markers to enable alignment of an image data set to the body of a patient.

FIG. 8 is a block diagram illustrating an example of a computing system to process the present technology.

### DETAILED DESCRIPTION

A technology is provided for using an augmented reality (AR) headset to enable one or more initial visible markers (e.g., optical codes) to be identified that are on a body of a person in view of a sensor of the AR headset (e.g., visible light sensor or IR sensor) during a medical procedure. An image data set can be aligned with a body of a person using one or more initial visible markers and image visible markers on or near the body of the person. Additional visible markers (e.g., optical codes) may be added to the body of the person and the alignment accuracy of the initial visible markers may be transferred to or applied to the additional visible markers. For example, the coordinate system of the one or more initial visible markers may be transferred to the one or more additional visible markers. This transfer may include the 3D coordinates of any objects in the 3D coordinate system as viewed by the AR headset.

This transfer may occur because the initial visible markers may be expected to have diminished accuracy or capability for alignment during a medical procedure for the alignment of the image data set to the body of the person. In addition, the initial visible markers may have diminished accuracy for expected or unexpected reasons. For example, if an initial visible marker moves (e.g., due to an incision), becomes partially covered by a surgical drape or is partially obscured by fluid, then the alignment of the image data set provided by the initial visible markers is less useful. Thus, alignment may be transferred to additional visible markers identified after the initial alignment phase or at another time during the medical procedure. These additional visible markers are not expected to have diminished alignment accuracy and can maintain the alignment of an image data set with the body of a person or patient through an AR headset.

The initial visible markers and additional visible markers may be optical codes, visible icons, visible characters or numbers, or other visible markers that can be detected using an AR headset. These initial visible markers and additional visible markers may be registrable image alignment points. As will be described later, the initial visible markers may have associated image visible markers that are viewable in an image data set (e.g., a CT scan) and are a fixed and known distance from the initial visible codes. In another configuration, the image visible markers may be co-incident with the initial visible markers, or the image visible markers may be formed into the initial visible markers. For example, the image visible marker(s) may be etched into, cut into or formed onto radiopaque metal (e.g., titanium) or other radiopaque material. In another example, the image visible marker may be a radiopaque marker that has a visible marker aspect defined by forming the visible marker in a visually detectable shape (e.g., a cross, icon, character, alpha numeric value, image, or other shape). Because the shape is formed from radiopaque materials, the visible marker is combined with an image visible marker and is optically visible to the AR headset and also visible in an image data set (e.g., a CT scan, MRI, etc.).

This technology may be viewed as transferring the positional accuracy of one or more initial visible markers to one or more additional visible markers during a medical procedure. More specifically, the coordinate system of the one or more initial visible markers can be transferred to the one or more additional visible markers. Alternatively, the additional visible markers can be inserted into the coordinate system occupied by the initial visible markers. For example, the location of the additional visible markers, as viewed through the AR headset, may be learned with respect to an image data set. The additional visible markers may be correlated to the initial visible markers that include image visible markers, which have an already known relationship with respect to the image data set. In one example, the transfer of accuracy may optionally occur after the initial visible markers have been used for alignment of the image data set with the patient or persons' body through an AR headset. In another example, the transfer of accuracy may occur at any time after a patient enters into an operating room or after a patient receives anesthesia.

The transfer of alignment accuracy may be in preparation for the initial visible markers losing their alignment accuracy to align the image data set with a body of the person. For example, the alignment accuracy may be reduced or lost when the initial visible markers are moved, covered, obscured, etc. during a medical procedure. The positional accuracy of the initial visible markers (e.g., on the skin of the person or patient) before their alignment accuracy is reduced may be transferred to additional visible markers (e.g., supplemental visible markers) that are not expected to lose alignment accuracy for the image data set. For example, the additional visible markers may be placed on: pins, bone pins, or on the patient's skin where movement is expected to be minimal or movement is not expected to occur. As a result, the alignment accuracy or precision of the initial visible markers can be transferred to the additional visible markers (e.g., new visible markers) and then the initial visible markers (e.g. on the skin) or other less useful codes for alignment can be de-emphasized or disabled. The additional visible markers may then be emphasized and/or used for aligning the image data with the body of the person. The emphasis or de-emphasis of the visible markers may be performed by weighting the alignment contributions of the initial visible markers and additional visible markers depending on the expected usefulness of the optical code in aligning the image data set with the body of the person.

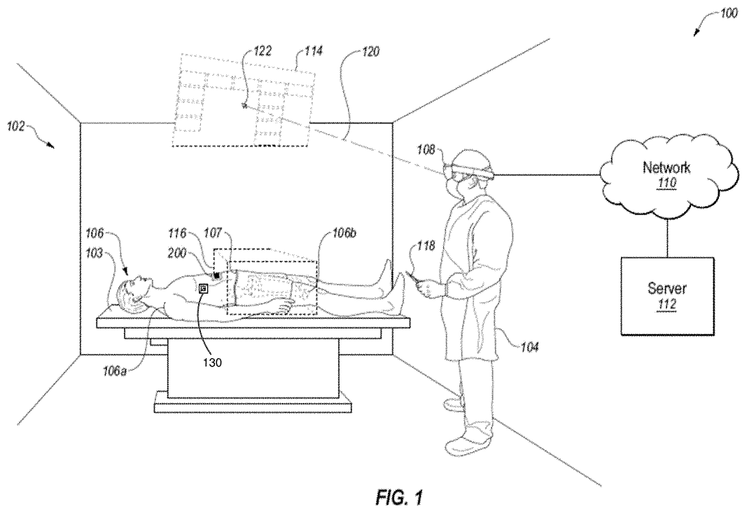

FIG. 1 illustrates an example augmented reality (AR) environment 100 in which an image data set of a patient 106 or body of a person may be aligned with actual views of the patient 106 using an initial visible marker 200 and image visible marker affixed to the patient 106. The environment 100 may include a physical space 102 (e.g., operating theater, a lab, etc.), a user 104, the patient 106, multiple initial visible markers 200 on the patient, a medical implement 118, and an AR headset 108 in communication with a server 112 over a computer network 110. A virtual user interface 114 and a virtual cursor 122 are also shown in dashed lines to indicate that these virtual elements are generated by the AR headset 108 and are viewable by the user 104 through the AR headset 108.

The AR headset 108 may be an AR computing system that is capable of augmenting actual views of the patient 106 with an image data set 116. For example, the AR headset 108 may be employed by the user 104 or medical professional in order to augment actual views of the patient 106 with one or more 3D image data set views or radiologic images of the patient 106 including, but not limited to, bones 106b, cartilage, muscles, organs, or fluids. The AR headset 108 may allow an image data set 116 (or a projection of the image data set) to be dynamically reconstructed. So, as the user 104 moves around the patient 106, the sensors of the AR headset 108 determine the location of the user 104 relative to the patient 106, and the internal anatomy of the patient displayed using the image data set can be reconstructed dynamically as the user chooses different orientations relative to the patient. For example, the user 104 may walk around the patient 106. Then the AR headset 108 may augment actual views of the patient 106 with one or more acquired 3D radiology images or image data sets 116 (MRI, CT scan, etc.) of the patient 106, so that both the patient 106 and the image data set 116 of the patient 106 may be viewed by the user 104 from any angle (e.g., a projected image or a slice from the image data set may also be displayed). The AR headset 108 may be a modified version of the Microsoft HOLOLENS, Meta Company META 2, Epson MOVERIO, Garmin VARIA VISION or other AR headsets.

The image data set or 3D radiology images may be a previously acquired image(s) of a portion of body of a person using a non-optical imaging modality (e.g., using MRI (magnetic resonance imaging), CT (computed tomography) scanning, X-ray, etc.). The image data set can be aligned to the body of the person using an image visible marker that is a fixed distance from at least one visible marker located on the body of the person. For example, an image visible marker and a visible marker (e.g., an optical code, an AprilTag or 2D optical bar code) may both be attached onto one piece of material (e.g., co-located or in fixed proximity of each other) to facilitate the alignment of the image data set with the body of the person. A medical professional may view a virtual interior of the patient using the image data set 116, while looking at the actual patient through an AR headset 108.

An image visible marker is a marker that can be viewed in a non-visible imaging modality, such as a captured radiology image or an image data set, which may not be optically visible to the AR headset. The image data set may be captured with a representation of the image visible marker using machine captured images that capture structures of the human body with the non-optical imaging modality. The representation of the image visible marker in the image data set may be aligned with the body of the patient using the known fixed position of the image visible marker with respect to the one or more visible markers affixed on the body of the person (as described in further detail later). For example, the image visible marker may be a radiopaque marker, an MRI bead, an echogenic structure for ultrasound, etc.

At least one of the visible markers on the body of the person can have a fixed position relative to an image visible marker. This allows an image data set (e.g., a radiology image) to be aligned to the body of the person using a fixed distance between the image visible marker and the one or more visible markers on the body of the person, as viewed through an AR display (e.g., an AR headset).

Initial visible marker(s) 200 may be affixed to the patient 106 prior to the generation of image data of the patient 106 (e.g., capture of the MRI, CT scan, X-ray, etc.), and then remain affixed to the patient 106 while the patient 106 is being viewed by a user 104 through the AR headset 108. Then, the initial visible marker 200 and image visible marker may be employed by the AR headset 108 to automatically align the image data set of the patient 106 with actual views of the patient 106. Further, employing the initial visible marker 200 used during the capturing of the image data to automatically retrieve the image data may ensure that the image data set 116 retrieved by the AR headset 108 matches the actual patient 106 being viewed through the AR headset 108.

Additional visible markers 130 may be added after the initial alignment of the image data set with the body of the person or after a patient has been brought into an operating room. For example, an additional visible marker 130 may be added to a body of a person on a skin location where the skin is not expected to move during a medical procedure. For example, the additional visible marker 130 may be added to skin over the ribs or sternum which are not expected to move during the medical procedure. An incision in the skin or other bodily movement may cause the initial visible markers 200 to move and the additional visible markers 130 are provided to supply alignment for the image data set despite movement of the initial visible markers or decreased alignment ability of the initial visible markers 200. In another example, the additional visible marker 130 may be affixed to a pin that can be inserted into bone or other tissue in order to reduce movement of the additional visible marker 130.

The alignment accuracy of the initial visible marker 200 may be transferred to the additional visible marker 130. This transfer may occur because the initial visible marker 200 may be expected to have diminished capability for alignment during the medical procedure or at some point after the initial alignment of the image data set to the body of the person. For example, if an initial visible marker 200 moves (e.g., due to an incision), becomes partially covered or is partially obscured by fluid, then the alignment of the image data set provided by the initial visible marker 200 may be less useful or less aligned. Thus, alignment may be transferred to an additional visible marker 130 identified after the initial alignment phase or at another time during the medical procedure but after the initial image data set capture (e.g., a CT scan capture). For example, the coordinate system of the one or more initial visible markers can be transferred to the one or more additional visible markers along with the positions of any objects in the 3D coordinate system viewable by the AR headset. Alternatively, the additional visible markers can be inserted into the 3D coordinate system of the initial visible markers. More specifically, when the position of an additional visible marker 130 is referenced to the position of an initial visible marker 200 and a related image visible marker during the transfer process, then a determination or measurement may be made as to a location that the image visible marker in the image data set can be aligned with respect the additional visible marker (e.g., new visible marker) to maintain alignment. This means the relationship between the additional visible markers and initial visible markers will be known, which in turn allows the image visible markers of the initial visible markers in the image data set to be aligned to the body of the person by then referencing the additional visible markers.

A virtual user interface 114 may be generated by the AR headset 108 and may include options for altering the display of the projected inner anatomy of the patient 106 from the image data set 116 of the patient 106. The virtual user interface 114 may include other information that may be useful to the user 104. For example, the virtual user interface 114 may include information about the patient or the medical implements 118 (e.g., medical instruments, implants, etc.) being identified with a visible marker. In another example, the virtual user interface 114 may include medical charts or other medical data of the patient 106. In some configurations, the image data set 116 or captured radiological data of a person may be displayed by the AR headset 108 using a volume of the image data set 116 to display radiologically captured anatomy (e.g., bones 106b, tissue, vessels, fluids, etc.) of the patient 106 from the image data. This image data may contain axial slices, coronal slices, sagittal slices, or oblique slices of the image data. Slices may be two-dimensional (2D) slices, three-dimensional (3D) slices, and/or four dimensional (4D) slices (3D images with a time sequence of images) that have a depth as well as a height and width (e.g., one or more layers of voxels). A user 104 may control the virtual user interface 114 using: hand gestures, voice commands, eye movements, remote controls (e.g., a finger clicker), a 3D mouse, a VR wand, finger sensors, haptic technology, or other control methods.

In one example configuration, multiple users each wearing an AR headset 108 may be simultaneously present to view the patient 106 augmented with image data of the patient 106. For example, there may be multiple AR headsets 108 that are used during medical procedures. One AR headset 108 may be used by a first medical professional to adjust and manipulate the radiological images being displayed to both AR headsets and the second head set may be used by a second medical professional to assist in performing the medical procedure on the patient. Additionally, one medical professional may be able to turn on or off the radiological image at the request of the other medical professional.

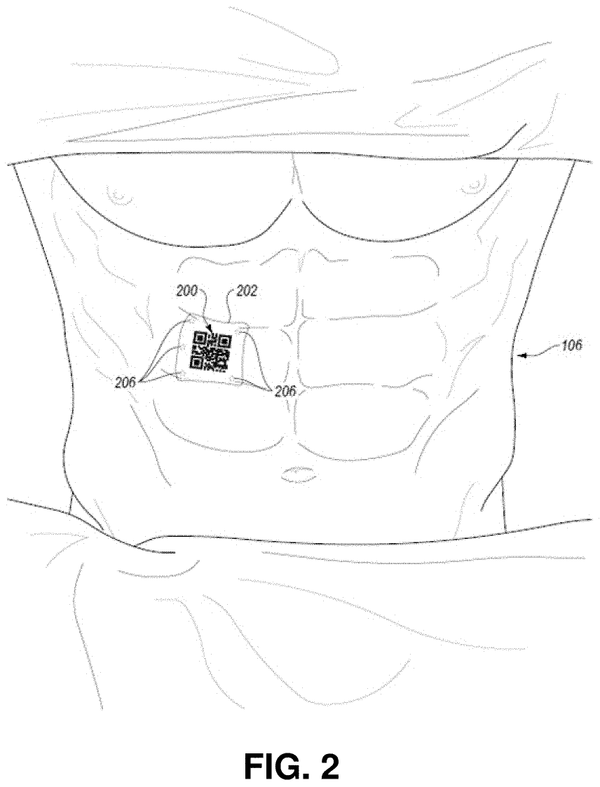

FIG. 2 illustrates a visible marker 200 of FIG. 1 that is an optical code affixed to the patient 106 of FIG. 1. With reference to both FIG. 1 and FIG. 2, the visible marker 200 may be perceptible to an optical sensor, such as an optical sensor built into the AR headset 108. In some embodiments, the visible marker 200 may be an optical code, AprilTag, a linear barcode, a matrix two-dimensional (2D) barcode, a Quick Response (QR) code, or some combination thereof. An AprilTag is type of two-dimensional bar code which may be a visual fiducial system which is useful for augmented reality and camera calibration. The AprilTags may be used to compute the 3D position, orientation, and identity of the tags relative to a camera, sensor, or AR headset.

The visible marker 200 may be linked to medical data of the patient 106 such that the medical data of the patient 106 can be accessed with the visible marker 200. For example, the visible marker 200 may be used to automatically retrieve the image data set to be used in a medical procedure for the patient using the AR system.

The visible marker 200 may further be associated with markers or image visible markers 206 that are perceptible to a non-optical imaging modality. Examples of a non-optical imaging modality may include, but are not limited to, an MRI modality, a Computerized Tomography (CT) scan modality, an X-ray modality, a Positron Emission Tomography (PET) modality, an ultrasound modality, a fluorescence modality, an Infrared Thermography (IRT) modality, 3D Mammography, or a Single-Photon Emission Computed Tomography (SPECT) scan modality. In another example, the non-optical images or image data sets may be an image or image data set which includes a combination of two or more forms of non-optical imaging modality as listed above (e.g., two or more images combined together, combined segments of two or more non-optical images, a CT image fused with an MRI image, etc.). Each image data set in a separate modality may have an image visible marker 206 in the individual image data set which may allow a PET image, a CT image, an MRI image, a fluoroscopy image, etc., to be aligned and referenced together with a visible marker on a body of a person in an AR system view. Forming the image visible markers 206 from a material that is perceptible to a non-optical imaging modality may enable the image visible markers 206 to appear in an image data set of the patient 106 that is captured using a non-optical imaging modality. Examples of image visible markers 206 include, but are not limited to: metal spheres, liquid spheres, radiopaque plastic, metal impregnated rubber, metal strips, paramagnetic material, and sections of metallic ink.

The image visible markers 206 may be arranged in a pattern and may have a fixed position relative to a position of the initial visible marker 200. For example, in the embodiment disclosed in FIG. 2, the initial visible marker 200 may be printed on a material 202 (such as an adhesive bandage, paper, plastic, metal foil, etc.) and the image visible markers 206 may be affixed to the material 202 (e.g., embedded in the material 202 and not visible on any surface of the bandage). In this embodiment, the image visible markers 206 may be arranged in a pattern that has a fixed position relative to a position of the visible marker 200 by being arranged in the fixed pattern in the bandage or material 202. Alternatively, the image visible markers 206 may be embedded within the visible marker 200 itself, such as where the image visible markers 206 are embedded within an ink with which at least some portion the visible marker 200 is printed on the material 202 and the ink includes a material that is perceptible to the non-optical imaging modality, such as ink particles that are radiopaque and are not transparent to X-rays. In these embodiments, the visible marker 200 itself may serve both as a visible marker and as the pattern of markers. Additionally, the image visible markers 206 may be arranged by affixing or printing (at least temporarily) the visible marker 200 directly on the skin 106a of the patient 106. By arranging the image visible markers 206 in a pattern that has a fixed position relative to a position of the visible marker 200, this fixed position may be employed to calculate the location of the pattern of the image visible markers 206 with respect to a visible location of the visible marker 200, even where the image visible markers 206 are not themselves visible or perceptible to sensors of the AR headset 108.

Once the visible marker 200 and the image visible markers 206 are affixed to the patient 106 in a fixed pattern, the non-optical imaging modality (to which the image visible markers 206 are perceptible) may be employed to capture image data of the patient 106 and of the image visible markers 206. In particular, the image data may include internal anatomy (such as bones 106b, muscles, organs, or fluids) of the patient 106, as well as including the pattern of image visible markers 206 in a fixed position relative to the positions of the inner anatomy of the patient 106. In other words, not only will the internal anatomy of the patient 106 appear in the image data of the patient 106, but the image visible markers 206 will also appear in the image data set of the patient 106 in a fixed pattern, and the position of this fixed pattern of the image visible markers 206 will appear in the image data set in a fixed position relative to the positions of the internal anatomy of the patient 106. In one example, where the non-optical imaging modality is a CT scan modality, the CT scan images may display the bones 106b, organs, and soft tissues of the patient 106, as well as the image visible markers 206 arranged in a fixed position with respect to the positions of the bones 106b, organs, and soft tissues of the patient 106.

Further, the patient 106 may be moved, for example, from a medical imaging room in a hospital to an operating room in the hospital. Then a user 104 (such as a medical professional) may employ the AR headset 108 to determine a location of the visible marker 200 on the body of a person or patient. Next, the AR headset 108 may automatically retrieve the image data of the patient 106 based on the visible marker.

After detecting the visible marker 200 (e.g., an initial visible marker) in the 3D space 102, the AR headset 108 may automatically calculate the position of the pattern of the image visible markers 206 in the 3D space 102 and with respect to one another. This automatic calculation may be based on the sensed position of the visible marker 200 in the 3D space 102 and may also be based on the known fixed position of the pattern of the image visible markers 206 relative to the position of the visible marker 200. Even where the image visible markers 206 are not perceptible to the AR headset 108 (for example, due to the image visible markers 206 being embedded or underneath a material), the AR headset 108 can automatically calculate the location of the pattern of the image visible markers 206 based on the position of the visible marker 200 and on the fixed position of the pattern of the image visible markers 206 relative to the position of the visible marker 200. In this example, these fixed positions may enable the AR headset 108 to automatically calculate the position of the pattern of the image visible markers 206 in the 3D space 102 with respect to one another even where the AR headset 108 is not directly sensing the positions of the image visible markers 206.

After calculating the location of the pattern of the image visible markers 206 or image visible markers in the 3D space 102, the AR headset 108 may then register the position of the internal anatomy of the patient 106 in the 3D space 102 by aligning the calculated position of the pattern of the image visible markers 206 in the 3D space 102 with the position of the pattern of the image visible markers 206 in the image data set. The alignment may be performed based on the calculated position of the pattern of the image visible markers 206 in the 3D space 102 and the fixed position of the image data set to the image visible markers 206 relative to the positions of the internal anatomy of the patient 106. This alignment and registration may then enable the AR headset 108 to display in real-time the internal anatomy of the patient 106 from the image data projected onto actual views of the patient 106.

Thus, the visible marker 200, and the associated pattern of the image visible markers 206, may be employed by the AR headset 108 to automatically align the image data of the patient 106 with actual views of the patient 106. Further, one or more visible markers 200 (e.g., an optical code, an AprilTag and 2D bar code or another combination of visible markers) may be employed to automatically retrieve the image data obtained during the capturing of the image data may ensure that the image data retrieved by the AR headset 108 matches the actual patient 106 being viewed through the AR headset 108.

In a further example, multiple visible markers 200 may be simultaneously affixed to the patient 106 in order to further ensure accurate alignment of image data of the patient 106 with actual views of the patient 106 in the 3D space 102. Also, the pattern of five image visible markers 206 disclosed in FIG. 2 may be replaced with another pattern, such as a pattern of three markers or a pattern of seven markers and each visible marker may have a different pattern. Further, since the image visible markers 206 are affixed to an outside layer of the patient 106, the image visible markers 206 may not all be in one plane, but instead may conform to any curvatures of the outside layer of the patient 106. In these embodiments, the fixed position of the pattern of the image visible markers 206 relative to a position of the visible marker 200 may be established after affixing the visible marker 200 and the image visible markers 206 to the patient 106 to account for any curvatures on the outside layer of the patient 106.

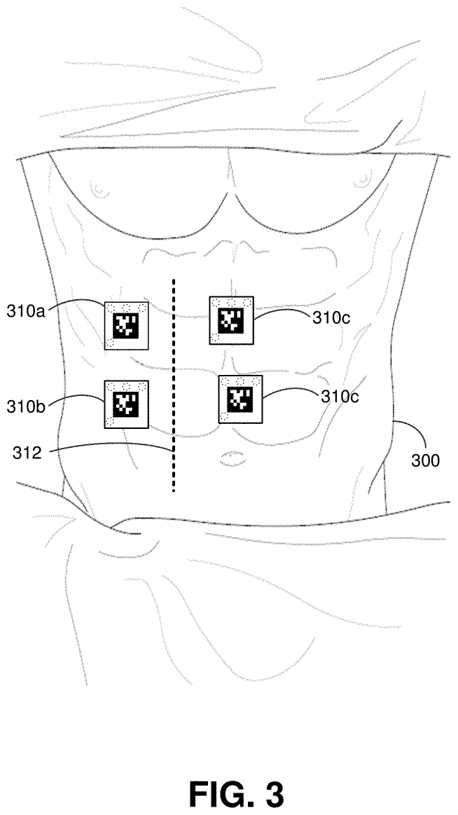

FIG. 3 illustrates a real world view that may be seen through the AR headset or AR system or also seen using human vision. The actual view may include a body of a patient 300 and a plurality of visible markers affixed to the patient.

As discussed earlier, the initial visible markers 310a-d affixed to the patient 300 can be used to identify a position and orientation of the body of the patient within the 3D coordinate space visible through the AR headset. This position and orientation information can be tracked for each visible marker and can be used when determining the position and orientation of the image data set to be aligned with the body of the person. The initial visible markers 310a-d may be captured and registered using the AR headset while viewing the patient during a medical procedure. Each of the initial visible markers 310a-d may be located in a fixed position relative to an image visible marker as illustrated in FIG. 2. In addition, the initial visible markers 310a-d can be used to reference previously captured radiological images of the patient to a body of the patient viewed through an augmented reality display (e.g., an AR headset). These initial visible markers 310a-d may also be near a desired surgical site 312 where an incision may be made.

The image data set may be aligned with the body of the person using one or more initial visible markers on the body of the person. The initial visible markers may be viewed through the AR headset and the fixed position of the image visible marker with respect to the initial may be referenced to a representation of the image visible marker in the image data set.

In one configuration, the system can automatically detect that the initial visible markers 310 a-d are: moving due an incision, on a moving body part or on moving skin which may have pulled the initial visible markers apart from each other. As a result, the system may reduce the weights for the moved initial visible markers 310 a-d and boost the weights for the additional visible markers (e.g., the newly added codes) for alignment purposes. Alternatively, the initial visible markers may be deactivated 310 a-d and not used for alignment purposes at all upon determining the initial visible markers 310 a-d have moved.

The location of one or more additional visible markers may be identified in the coordinate system of the initial visible markers. Additional visible markers may be added in preparation for changes that may occur to the initial visible markers during the medical procedure. The additional visible markers which have been added to the body of the person may be attached to at least one of: a bone of the body of the person, a bone pin placed in a bone of the body of the person, an organ, a blood vessel or an inner tissue of the body of the person. In addition, the additional visible markers may be: on the skin of the patient that is not expected to move, attached to an inner physical layer of the body of the person that is not expected to move, on a facial feature, on a unique body landmark, etc.

More specifically, these additional visible markers may be added to places on the skin of the patient where the additional visible markers are not expected to move, such as a location away from a surgical incision. The additional visible markers may also be attached to a bone pin that is placed into a bone of a patient once the patient is under anesthesia. An additional visible marker with a bone pin will not be expected to move when a medical procedure occurs, such as the creation of an incision in the patient's skin.

In one configuration, the coordinate system of the one or more initial visible markers may be transferred to the one or more additional visible markers. This transfer of the coordinate system from the initial visible markers to the additional visible markers may include the transfer of coordinates for objects or items in the coordinate system of the initial visible markers. For example, the specific coordinates for objects viewed by the AR headset such as: image visible markers that are a known distance from initial visible markers, medical instruments, the body of the patient, an operating table, medical professionals, medical machines (e.g., x-ray imaging machines) or other objects may be transferred to the additional visible markers. Alternatively, the additional medical codes may be inserted into the coordinate system of the initial visible markers and the initial visible markers may be disabled which may result in a similar alignment outcome.

The transfer of the coordinate system to the additional visible markers or the insertion of the additional visible markers into the coordinate system may occur after a notification is received. The notification may be received that the one or more initial visible markers have moved or are less effective. In one example, a notification may be received in software of the AR headset which detects that a relative distance between two visible markers on body of the person or on skin has changed. These the two visible markers may then be considered displaced visible markers. More specifically, the one or more initial visible markers may be de-emphasized and only the additional visible markers may be emphasized.

In another configuration, each initial visible marker and additional visible marker that is registered may be assigned a weighting and the weight assigned to the visible marker can determine the amount the visible marker's position contributes to aligning the image data set with the body of the person. The weighting may be set to allow the alignment to take place using each of the registered visible markers as averaged together or some other combination using a computed distribution (e.g., mean, centroid, a normal distribution, etc.). The weighting may also be based on the systems confidence that the visible marker is located at the identified location. If the visible marker is difficult to detect, is at an awkward angle or may have some other issue with the visible marker, then the weighting of the visible marker for alignment purposes may be decreased. The weightings may be an integer or floating point number that weights the contribution of the visible marker to the alignment. The weighting values may alternatively be a value between 0 and 1 (e.g., 0.8) that determines how much the location of the visible marker contributes to alignment at a defined point in time. Further, when registration is occurring and multiple registered visible markers are being viewed through the AR headset, the medical professional can look at the scene from different angles, then the image data set (e.g., virtual patient image) can be moved slightly to try to perform a best fit to the multiple registered visible markers.

In one example configuration, the AR system and/or software may automatically identify when one or more initial visible markers have diminished alignment accuracy (e.g., have moved) and a notification may be sent to a user that at least one of the initial visible markers has diminished alignment accuracy. In one example, a notification that the one or more visible markers have moved can be triggered by detecting that a relative distance between two visible markers on body of the person or on skin has changed. This may result in the two visible markers being designated as displaced visible markers. The displaced visible markers may also be identified due to a change in relative distance of the displaced visible marker (e.g., closer or farther) with respect to: another visible marker, a visible landmark on the body of the person, a visible anatomical feature of the body of the person, a visible facial feature, a visible bone protrusion, a visible tissue protrusion, and/or a visible body contour. An alignment weighting of displaced visible markers may also be decreased, which decreases an amount the displaced visible markers are referenced in aligning the image data set with the body of the person.

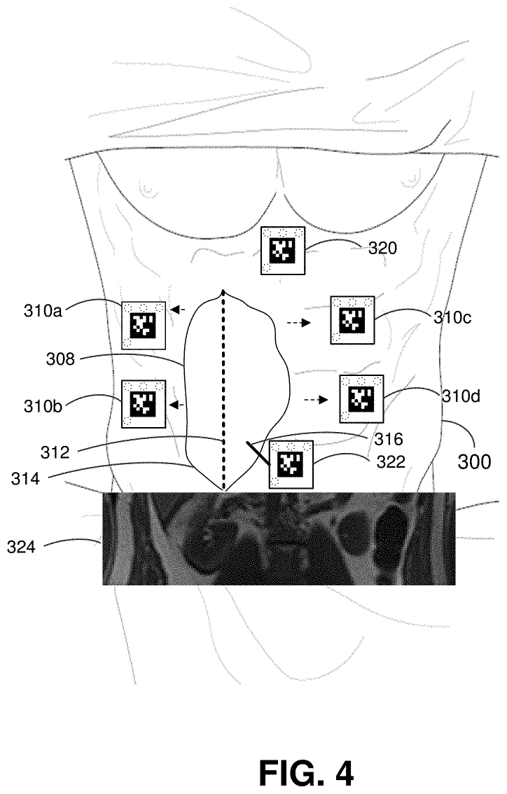

FIG. 4 illustrates that an incision may have caused one or more initial visible markers to move as an incision has caused the skin to draw back from the incision 308. For example, two initial visible markers 310a and 310b have moved to the left in the figure and two initial visible markers 310c and 310d may have moved to the right in the figure (as illustrated by the dotted arrows), as the skin of the patient has retracted from the incision site. Accordingly, these initial visible markers 310a-d may now be less reliable in aligning the image data set with the body of the patient.

Additional visible markers 320, 322 may have been added prior to the incision taking place but after the image data set 324 was captured. For example, an additional visible marker 320 may have been placed on the person's skin or body at a point on the person's body where an incision is not likely to impact the location of the additional visible marker significantly (e.g., a sternum). Alternatively, an additional visible marker 322 may be pinned 316 into the inner layers (e.g., bones) or inner organs of the patient and may have reduced movement as a result.

The capability for aligning the image data set with the body of the person may be transferred from the initial visible markers 310a-d to the additional visible markers 320, 322. As discussed, the coordinate system of the one or more initial visible markers may be transferred to the one or more additional visible markers. This transfer may be a complete transfer of alignment influence from the initial visible markers 310a-d to the additional visible markers 320, 322. Alternatively, the transfer may be a partial transfer of alignment influence for the alignment of the image data set with the body of the patient. For example, the weighting for the alignment influence may be 80% assigned to the additional visible markers and 20% assigned to the initial visible markers. Thus, the initial visible markers 310a-d and additional visible markers 320, 322 may be weighted according to the desired amount of contribution of the initial visible markers or additional visible markers toward the alignment of the image data set with the body of the person.

An alternative method may be used for aligning the image data set with a body of a person as viewed through the AR headset. In this method, an x-ray generated image of at least a portion of the body of the person represented in the image data set is obtained or identified. The image data set may be aligned to the x-ray generated image by using data fitting to align identified anatomical structures that are shared in the image data set and the x-ray generated image. For example, the same bones in both the image data set and x-ray image may be aligned. The details of the data fitting are described in further detail in the U.S. patent application entitled IMAGE DATA SET ALIGNMENT FOR AN AR HEADSET USING ANATOMIC STRUCTURES AND DATA FITTING with U.S. Ser. No. 17/536,009 as filed Nov. 27, 2021 which is incorporated by reference in its entirety herein.

The aligning of the image data set and the x-ray generated image with patient anatomy viewable through the AR headset may enable identification of an initial visible marker formed in a radiopaque marker that is represented in the x-ray generated image. This initial visible marker may be referenced to the visible marker formed in the radiopaque marker visible on the patient. The image visible marker for the initial visible marker may be copied from the x-ray generated image to the image data set, and the image data set can be aligned with the body of the patient by aligning the initial visible marker with an image visible code with an initial visible marker and image visible marker that is visible on the patient. The image visible marker may be a metal marker or titanium marker with the visible marker formed in the metal marker.

In another configuration, one or more additional visible markers 320, 322 may be registered in a coordinate system of the one or more initial visible markers. These additional visible markers may have a bone pin to attach or fix the additional visible markers to bones or the additional visible markers may be attached to skin or other soft tissue that is not expected to move.

A notification may be received that one or more initial visible markers have moved. A relative distance between two visible markers on an outer layer of the body of the person or skin may be detected as having changed. Alternatively, a user interface control message may be received from a user that a position of the visible markers has changed due to movement of the skin or some other issue has occurred that makes the initial visible markers less valuable or less usable in the alignment process.

The use of the additional visible markers may be emphasized to maintain alignment of the image data set with the body of the person. By comparison, the displaced visible markers may be de-emphasized or terminated for aligning the image data set. For example, the initial visible markers which may be de-emphasized for use in alignment may be any visible markers which are not additional visible markers. The image data set with the body of the person may then be re-aligned using the one or more additional visible markers, which have been emphasized, as viewed through the AR headset. Emphasizing the use of the additional visible markers may also entail terminating the use of the displaced visible markers for purposes of aligning the image data set with the body of the person.

The initial visible markers affixed to the body of the patient or person may be visible markers that are expected to have a diminished accuracy to contribute to alignment during the medical procedure. This diminished accuracy for alignment may be due to: the initial visible markers have moved from their original position due to an incision in the skin, movement of the skin or movement of the patient's body part (e.g., the spine bending or twisting), or other changes.

The one or more additional visible markers may be registered in the system, and at least a portion of alignment influence for the image data set can be transferred to the one or more additional visible markers. The one or more additional visible markers (e.g., attached to bone, inner tissue or located outside the movement area to avoid the incision or outside the work area) may be emphasized in maintaining alignment of the image data set with the body of the person. For example, the one or more initial visible markers may be de-emphasized and only the additional visible markers may be emphasized.

The emphasizing of the additional visible marker may be done through increasing an alignment weighting for the additional visible marker added to the body of the person. In one case, increasing the weighting of the additional visible marker may be done after a user provides input that the additional visible marker is attached to an internal physical layer of the body of the person or is anchored to a bone using a bone pin.

In another example, during a medical procedure, the initial visible markers may be placed on or attached to a person's skin and then the image data set (e.g., CT scan) can be aligned to a body of a person. During surgery a medical professional or doctor may add additional visible markers that are attached to a bone pin, and the pin can be inserted into the bone (or other tissue) after the patient is under anesthesia and will not feel the pain of the bone pin being inserted into the bone and/or other tissue. The initial visible markers can then be de-emphasized or completely removed from use in the alignment process because they are not deemed to be as reliable as when they were initially placed on the body of the person. In the case of removing the initial visible markers from the alignment process, the alignment of the image data set can be transferred to the additional visible markers. For example, the initial visible markers may be less reliable for alignment when the skin is cut and the initial visible markers on the skin move and become unreliable for alignment. Accordingly, the image data set may then be realigned with the body of the person using the one or more additional visible markers that are emphasized for alignment.

Another use example may be that the patient has spinous processes or an iliac crest that may be exposed during a medical procedure. If multiple initial visible markers are placed on the skin, when a surgical incision is made to access the spinous processes or inner layers of the patient, then the visible markers may move out from their original position as the skin pulls away from the incision. Then the initial visible markers become far less useful in alignment of the image data set with the patient. Where a pre-operative CT scan has been captured, then the initial visible markers are fairly accurate for alignment and the initial visible markers do not generally move more than a few millimeters relative to each other. Once an incision is made and the skin is retracted, the codes are likely to move significantly (e.g. a few centimeters). At this point, the pre-operative CT scan may not have a useful alignment or the moved initial visible markers may reduce the accuracy of the alignment of the pre-operative CT scan.

As a result of this and similar issues, additional visible markers can be introduced into the area viewed by the AR headset during the medical procedure. The additional visible markers may be attached with a bone pin to a bone (e.g., to iliac crests, spinous processes, the skull, etc.). The additional visible markers can be anchored to bones or tissues that are not likely to move. Alternately, the additional visible markers may be placed at a location where the additional visible markers are far enough from the surgical field that the additional visible markers are not affected by surgical incision or other factors that might make the additional visible markers less useful for alignment of the image data set with the body of the person.

In one configuration, there may be a command that can be issued by a user of the AR headset that is a “learn” the location command for the additional visible markers. The command may be a menu option, a hand gesture, a voice command or other command that can be received by the AR headset and related software. This command may trigger the registration of the additional visible markers (e.g., new visible markers) and allow the AR headset to determine the relative position of the additional visible markers in the 3D coordinate system or transfer the 3D coordinate system to the additional visible markers. This identification of the additional visible markers may happen after an accurate registration using the initial visible markers.

The AR headset may also be generating a number of rays to use in identifying the visible markers. These may be rays between the corners of the visible markers to find the center of the visible markers or rays from the AR headset to the centers of the visible markers, etc. Once registration is complete then the coordinates of the additional visible markers in the original registration space may be fixed. Then after that the system can identify the additional visible markers and determine where they are in the 3D virtual image coordinate space. When the registration is accurate and enough rays have been computed to register the additional visible markers, the system may automatically look for or track the additional codes after the first stage of registration. Using the present system, the alignment accuracy will not be lost when the skin shifts through cutting but millimeter level accuracy is transferred to the additional visible markers to maintain the image data set registration accuracy.

The user can be provided with a user interface to select whether to de-emphasize or disable the initial codes on the skin of the patient. Then alignment occurs to the additional visible markers. If the patient moves, then the system is registering the CT scan to the additional codes and the accuracy is the same as existed for the initial visible markers.

In another example of diminished accuracy for alignment, a portion of the visible marker may be covered and may be less usable for alignment. This may include the visible marker being covered by a surgical drape fabric, a medical instrument, or some other object. In another example, a portion of the visible marker may not be recognizable due to bodily fluid (e.g., blood, lymph fluid, puss, etc.), tissue, bone fragments, or some other matter getting on the visible marker. This bodily fluid, such as blood or another fluid, may make a portion or all of the initial visible marker(s) difficult to detect.

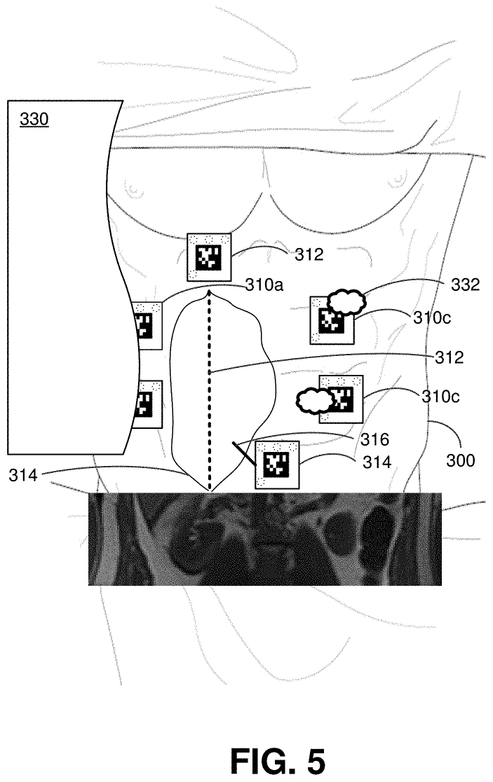

FIG. 5 illustrates that visible markers may have diminished accuracy for alignment or may be disabled by events that may occur during the medical procedure. As has been described, the skin moving due to an incision may cause the initial visible markers to have reduced value in the alignment process. In another example of diminished accuracy, the initial visible markers may be partially covered by a surgical drape fabric 330 as the procedure is taking place, because the surgical drape fabric may need to be moved for sanitary or other practical reasons. The initial visible markers may then have a portion or even all of the initial visible marker covered making the initial visible markers less reliable for alignment purposes. For example, if a corner of one of the initial optical is covered, then the center of the visible marker may be more difficult to find. As a result, the center of the initial visible marker might only be estimated. In such cases, the use of the initial visible marker may have a reduced weighting for alignment or the use of the visible marker may be terminated, and the alignment may be completely transferred to one or more additional visible markers. Similar issues may occur where an object or material 332 such as fluid, blood, bone fragments, tissue, instruments or other items cover the initial visible marker.

An example summary of the alignment process with the transferred accuracy will now be provided. The alignment process may include putting the initial visible markers on the patient. Before the procedure begins, a CT scan may be performed and the image visible code associated with the initial visible markers is captured in the image data set and identifies for the AR headset and alignment software where alignment of the image data set (hologram) should occur during the medical procedure. Next, additional visible markers are added to the patient during the medical procedure. For example, the additional visible marker may have a pin and the pin may be inserted into the bone after anesthesia of the patient has occurred. This means the pins are put into something relatively fixed in the patient. Alternately, the additional visible markers may be placed on the skin where the visible marker is not expected to move, be disturbed, be occluded or otherwise lose the visible markers usefulness for alignment.

The additional visible markers (i.e., new visible marker) coordinates are then learned by the AR system and software. This means the location of additional visible markers can be determined with respect to the initial visible markers and within the 3D coordinate system that is viewable by the AR headset. Once registration of the additional visible markers occurs, then the additional visible marker locations are known with respect to image visible marker and the image data set (e.g., CT scan) and the additional visible markers can then be used for alignment of the image data set with the body of the person. In other words, the alignment now occurs with the additional visible markers, and the additional visible markers (e.g., supplemental codes, anchored codes, unmodified codes, etc.) can be used for alignment of the CT scan.

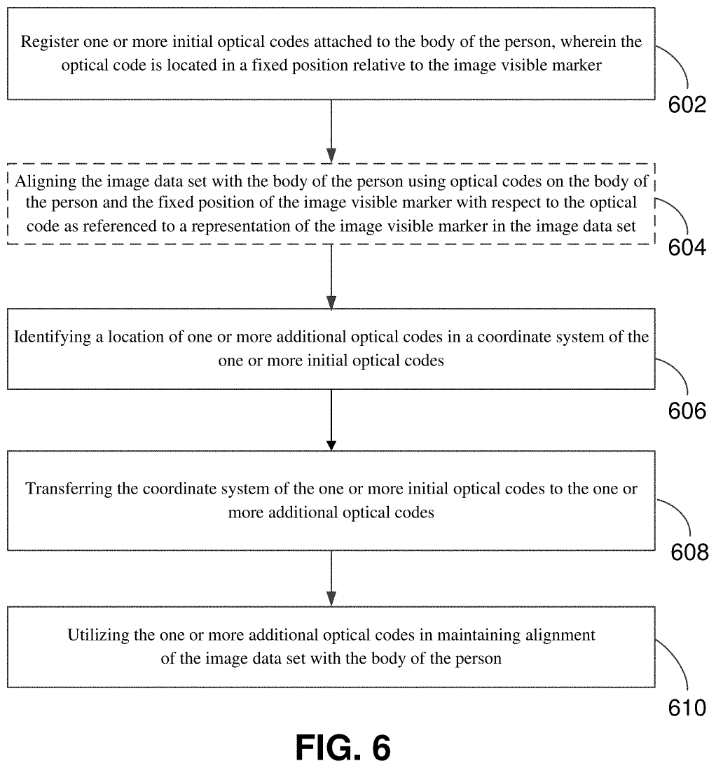

FIG. 6 is a flowchart of an example method for using an augmented reality (AR) headset to co-localize an image data set, containing an image visible marker, with a body of a person. In these and other embodiments, the method may be performed by one or more processors based on one or more computer-readable instructions stored on one or more non-transitory computer-readable media.

The method may register one or more initial visible markers attached to the body of the person, wherein the visible marker is located in a fixed position relative to the image visible marker, as in block 602. For example, a visible patient with a visible marker is registered or detected by a camera of the AR headset used by a medical professional.

Another optional operation may be aligning the image data set with the body of the person using one or more visible markers on the body of the person as viewed through the AR headset and the fixed position of the image visible marker with respect to the visible marker as referenced to a representation of the image visible marker in the image data set, as in block 604. The visible marker on a body of the person may be located in a fixed position relative to an image visible marker, as discussed earlier.

A location of one or more additional visible markers may be identified in a coordinate system of the one or more initial visible markers, as in block 606. Then one or more additional visible markers identified may have been added to the body of the person and anchored to an inner physical layer of the body of the person. For example, the additional visible markers may be anchored to one or more bones using a bone pin. The additional visible markers may be added before or after the first alignment of the image data set but after the image data set is initially captured.

Another operation may be transferring the coordinate system of the one or more initial visible markers to the one or more additional visible markers, as in block 608. This transfer of the coordinate system from the initial visible markers to the additional visible markers may include the transfer of coordinates for objects or items in the 3D coordinate system of the initial visible markers. For example, the specific coordinates for objects such as: image visible markers that are a known distance from visible markers, medical instruments, the body of the patient, an operating table, medical professionals, medical machines (e.g., x-ray imaging machines) or other objects may be transferred to the additional visible markers. Alternatively, the additional medical codes may be inserted into the coordinate system of the initial visible markers which may results in a similar outcome.

The transfer of the coordinate system to the additional visible markers or the insertion of the additional visible markers into the coordinate system may occur after a notification is received. The notification may be received that the one or more initial visible markers have moved. In one example, a notification may be received in software of the AR headset which detects that a relative distance between two visible markers on body of the person or on skin has changed. These the two visible markers may then be considered displaced visible markers. More specifically, the one or more initial visible markers may be de-emphasized and only the additional visible markers may be emphasized. Displaced visible markers may be identified due to a change in relative distance of the displaced visible marker with respect to at least one of: another visible marker, a visible landmark on the body of the person, a visible anatomical feature of the body of the person, a visible facial feature, a visible bone protrusion, a visible tissue protrusion, or a visible body contour.

In a sense, registering the one or more additional visible markers can include transferring at least a portion of alignment influence for the image data set to the one or more additional visible markers.

A further operation may be utilizing the one or more additional visible markers in maintaining alignment of the image data set with the body of the person, as in block 610. This may mean emphasizing or increasing an alignment weighting for an additional visible marker added to the body of the person after determining that the additional visible marker is attached to an internal physical layer of the body of the person. In further examples, the utilization of the additional visible markers may include emphasizing, de-emphasizing, terminating, or removing the use of the additional visible markers. The utilization of the additional visible markers may be varied by changing the weighting of the additional visible markers or using another existing prioritization method. In addition, the initial visible markers or initial codes that are displaced visual markers (e.g., displaced optical codes, displaced QR codes, displaced bar codes, etc.) may be de-emphasized or terminated from use in aligning the image data set. This means the initial visible markers that are displaced may be assigned a lower weighting or a zero weighting. In other words, decreasing an alignment weighting of displaced visible markers may decrease an amount the displaced visible markers influence alignment or are referenced in aligning the image data set with the body of the person. In some configuration, the utilization of the initial visible markers may include: emphasizing, de-emphasizing, terminating, or removing the use of the initial visible markers

Accordingly, the image data set may be re-aligned with the body of the person using the one or more additional visible markers that have been emphasized for alignment. The further alignment of the image data set with the body of the person may be viewed through the AR headset. In one example, the image data set of a radiology image can be presented at different levels of opacity depending on the needs of the user or medical professional. In addition, this opacity may be adjusted at any time.

In an additional configuration, the present technology may be used for a simulation of a medical procedure. The patient's body or patient's anatomy may be simulated using simulation structures. The simulation structures may be plastic or cadaver bones covered with soft material (plastics and rubbers to represent tissues, arteries, etc.) or other simulation materials. The simulated anatomy may include a visible marker and image visible code. Then image data set for the patient on which a procedure is to be performed in the future may be aligned with the simulated anatomy.

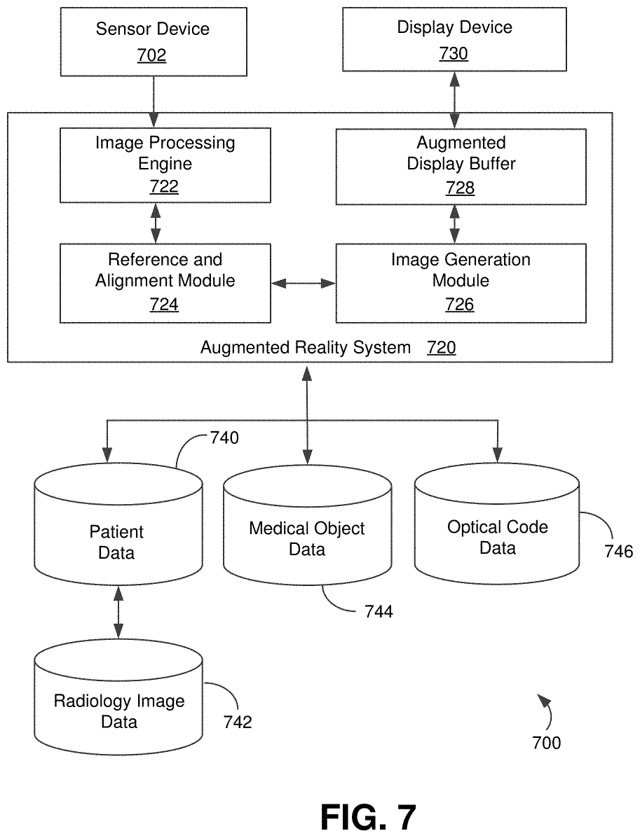

FIG. 7 illustrates an example system that may be employed to align an image data set and a patient's body using visible markers, as viewed through the AR headset. The system 700 may include a sensor device 702, an augmented reality system 720, a display device 730, and a plurality of databases. The system 700 may also include one or more processors, a memory, a file system, a communication unit, an operating system, and a user interface, which may be communicatively coupled together. In some embodiments, the system 700 may be, for example, a desktop computer, a server computer, a laptop computer, a smartphone, a tablet computer, an embedded computer, an AR headset, a VR headset, etc.

The sensor device 702 can be configured to capture visible data or IR data. In one example, the sensor device 702 may be used to capture visible data or IR data during a medical procedure. The visible data captured by the sensor device 702 may include images of a body of a person (or a portion of a body) and one or more medical implements (e.g., medical instruments, implants, and so on). The sensor device 702 may transmit the captured optical data or IR data to the augmented reality system 720. The system also may include surface sensors, optical sensors, infrared sensors, Lidar sensors or other sensors to detect and assist with mapping a real view or actual view detected by the AR system. Any object or surface may be detected for an operating theater, a patient, a room, physical geometry, medical implements, or any other physical surroundings or objects.

The augmented reality system 720 may include an image processing engine 722, a reference and alignment module 724, an image generation module 726, and an augmented display buffer 728. For example, the image processing engine 722 may receive the captured visible image data from the sensor device 702 and analyzes the visible image data or IR data to identify one or more visible markers, objects or people in the visible or IR image data. A plurality of different techniques may be used to identify objects within the visible or IR image data including but not limited to feature extraction, segmentation, and/or object detection.

The image processing engine 722 also identifies visible markers that may be affixed to both bodies of patients within the image and medical implements within the visible image data. Once the image processing engine 722 identifies an visible marker (e.g., an optical code, an AprilTag, a bar code, a QR code, and so on) the image processing engine 722 accesses the visible marker database 746 to retrieve information associated with the visible marker. In some examples, the visible marker is associated with a particular patient, a particular procedure, or a particular object. The visible markers may be used to more accurately identify the position and orientation of a medical implement, a body or a fluoroscopic device.

In some embodiments, the reference and alignment module 724 engages with the image processing engine 722 to reference and/or align any identified visible markers, a body of a person and an image data set with respect to each other. In addition, the reference and alignment module 724 can use visible marker information in the medical object data 744 to properly identify the patient, medical procedures to be performed, medical devices, etc. Once the position and orientation of body of the patient, visible markers and/or the image data set are determined relative to each other, the reference and alignment controller 726 can align any associated radiology images in the radiology image database 742 with both the body of the patient. In some examples, the radiology images are received from a radiology image database 742 based on patient records in a patient database 740.

The image generation module 726 can generate graphical data, virtual tools, a 3D surgical tract, 3D colorization or shading of a mass or organ, or highlighting of a mass, organ or target to display in a display device 730 as layered on top of the body of the patient or a medical implement. In some examples, this information can be loaded into an augmented display buffer 1028. This information can then be transmitted to a display device 730 for display to a user.

In one example, the patient database 740 includes a plurality of patient records. Each patient record can include one or more medical procedures to be performed on a patient. The patient records may also include notes, instructions or plans for a medical procedure. A patient record can also be associated with one or more radiology images in the radiology image database 742. In some examples, the radiological images include a representation of the image visible marker that allows the reference and alignment module 726 to properly align the image data set with the body of a patient using the fixed position of a visible marker with respect to the image visible marker. In some examples, the medical object data 744 includes information describing the medical implements, including medical instruments, implants, and other objects.

In some embodiments, the augmented reality system may be located on a server and may be any computer system capable of functioning in connection with an AR headset or display device 730. In some embodiments, the server may be configured to communicate via a computer network with the AR headset in order to convey image data to, or receive data from, the AR headset.

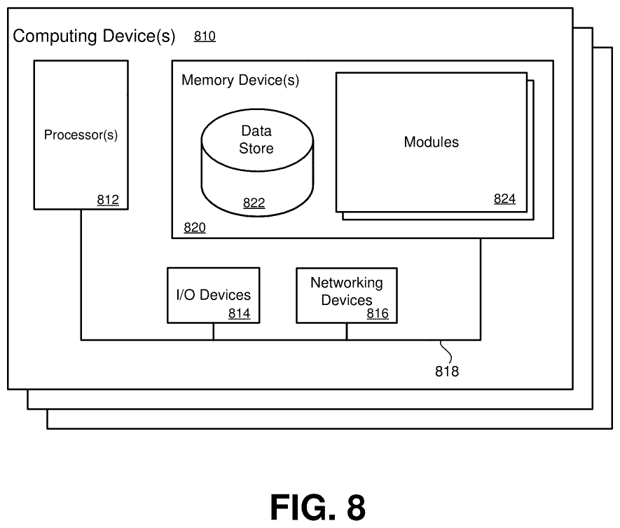

FIG. 8 illustrates a computing device 810 on which modules of this technology may execute. A computing device 810 is illustrated on which a high level example of the technology may be executed. The computing device 810 may include one or more processors 812 that are in communication with memory devices 820. The computing device may include a local interface 818 for the components in the computing device. For example, the local communication interface may be a local data bus and/or any related address or control busses as may be desired.

The memory device 820 may contain modules 824 that are executable by the processor(s) 812 and data for the modules 824. The modules 824 may execute the functions described earlier. A data store 822 may also be located in the memory device 820 for storing data related to the modules 824 and other applications along with an operating system that is executable by the processor(s) 812.

Other applications may also be stored in the memory device 820 and may be executable by the processor(s) 812. Components or modules discussed in this description that may be implemented in the form of software using high programming level languages that are compiled, interpreted or executed using a hybrid of the methods.

The computing device may also have access to I/O (input/output) devices 814 that are usable by the computing devices. An example of an I/O device is a display screen that is available to display output from the computing devices. Other known I/O device may be used with the computing device as desired. Networking devices 816 and similar communication devices may be included in the computing device. The networking devices 816 may be wired or wireless networking devices that connect to the internet, a LAN, WAN, or other computing network.

The components or modules that are shown as being stored in the memory device 820 may be executed by the processor 812. The term “executable” may mean a program file that is in a form that may be executed by a processor 812. For example, a program in a higher level language may be compiled into machine code in a format that may be loaded into a random access portion of the memory device 820 and executed by the processor 812, or source code may be loaded by another executable program and interpreted to generate instructions in a random access portion of the memory to be executed by a processor. The executable program may be stored in any portion or component of the memory device 820. For example, the memory device 820 may be random access memory (RAM), read only memory (ROM), flash memory, a solid state drive, memory card, a hard drive, optical disk, floppy disk, magnetic tape, or any other memory components.

The processor 812 may represent multiple processors and the memory 812 may represent multiple memory units that operate in parallel to the processing circuits. This may provide parallel processing channels for the processes and data in the system. The local interface 818 may be used as a network to facilitate communication between any of the multiple processors and multiple memories. The local interface 818 may use additional systems designed for coordinating communication such as load balancing, bulk data transfer, and similar systems.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A method for using an augmented reality (AR) headset to co-localize an image data set with a body of a person, comprising:
registering one or more initial visible markers attached to the body of the person, wherein the initial visible marker is located in a fixed position relative to an image visible marker;
identifying a location of one or more additional visible markers in a coordinate system of one or more initial visible markers;
transferring the coordinate system of the one or more initial visible markers to the one or more additional visible markers; and
utilizing the one or more additional visible markers in aligning the image data set with the body of the person.

2. The method as in claim 1, further comprising identifying that the one or more initial visible markers have diminished accuracy for alignment of the image data set.

3. The method as in claim 2, wherein the one or more initial visible markers with a diminished accuracy for alignment are at least one of: one or more initial visible markers which have moved, one or more initial visible markers which potentially will move, a portion of one or more initial visible markers is covered, or a portion of the one or more initial visible markers is not recognizable.

4. The method as in claim 1, wherein utilizing the one or more additional visible markers further comprises utilizing the additional visible markers by at least one of: emphasizing, de-emphasizing, terminating, or removing the use of the additional visible markers.

5. The method as in claim 1, further comprising at least one of: de-emphasizing, terminating, removing, or emphasizing the use of the initial visible markers in aligning the image data set.

6. The method as in claim 1, further comprising aligning the image data set with the body of the person using one or more visible markers on the body of the person as viewed through the AR headset and the fixed position of the image visible marker with respect to the visible marker as referenced to a representation of the image visible marker in the image data set.

7. The method as in claim 1, further comprises receiving a notification that the one or more initial visible markers have moved upon detecting that a relative distance between two visible markers on body of the person or on skin has changed, wherein the two visible markers become displaced visible markers.

8. The method of claim 7, further comprising de-emphasizing or terminating use of the displaced visible markers for aligning the image data set.

9. The method as in claim 1, further comprising de-emphasizing the one or more initial visible markers and emphasizing only the additional visible markers.

10. The method of claim 1, further comprising re-aligning the image data set with the body of the person using the one or more additional visible markers that have been emphasized for alignment.

11. The method of claim 1, further comprising identifying one or more additional visible markers which have been added to the body of the person and anchored to an inner physical layer of the body of the person.

12. The method of claim 11, further comprising increasing an alignment weighting for an additional visible marker added to the body of the person after determining that the additional visible marker is attached to the inner physical layer of the body of the person.

13. The method as in claim 7, further comprising decreasing an alignment weighting of displaced visible markers which decreases an amount the displaced visible markers are referenced in aligning the image data set with the body of the person.

14. The method of claim 7, further comprising identifying displaced visible markers due to a change in relative distance of the displaced visible marker with respect to at least one of: another visible marker, a visible landmark on the body of the person, a visible anatomical feature of the body of the person, a visible facial feature, a visible bone protrusion, a visible tissue protrusion, or a visible body contour.

15. The method as in claim 1, wherein registering the one or more additional visible markers includes transferring at least a portion of alignment influence for the image data set to the one or more additional visible markers.

16. A method for using an augmented reality (AR) headset to co-localize an image data set with a body of a person as viewed through the AR headset, comprising:
obtaining an x-ray generated image of at least a portion of the body of the person represented in the image data set;
aligning the image data set to the x-ray generated image by using data fitting to align identified anatomical structures in the image data set and the x-ray generated image;
registering one or more additional optical codes in a coordinate system of one or more initial optical codes;
receiving a notification that one or more initial optical codes have moved; and
utilizing the additional optical codes to maintain alignment of the image data set with the body of the person.

17. The method as in claim 16, wherein receiving a notification that the one or more initial optical codes have moved is performed by either:
detecting that a relative distance between two optical codes on an outer layer of the body of the person or skin has changed, wherein the two optical codes become displaced optical codes; or
receiving a user interface control message from a user that a position of the optical codes has changed due to movement of the skin.

18. The method of claim 16, wherein utilizing the additional optical codes further comprises emphasizing use of the additional optical codes.

19. The method of claim 16, further comprising aligning the image data set and x-ray generated image with patient anatomy viewable through the AR headset using an initial optical code formed in a radiopaque marker represented in the x-ray generated image as referenced to the initial optical code formed in the radiopaque marker visible on the body.

20. The method of claim 17, further comprising de-emphasizing or terminating use of the displaced optical codes for aligning the image data set.

21. The method as in claim 16, further comprising de-emphasizing initial optical codes for use in alignment which are not additional optical codes.

22. The method of claim 17, further comprising re-aligning the image data set with the body of the person using the one or more additional optical codes, which have been emphasized, as viewed through the AR headset.

23. The method of claim 16, further comprising identifying one or more additional optical codes which have been added to the body of the person and anchored to an inner layer of the body of the person.

24. The method of claim 16, further comprising identifying an additional optical code attached to at least one of: a bone of the body of the person, a bone pin placed in a bone of the body of the person, an organ, a blood vessel or an inner tissue of the body of the person.

25. A system for using an augmented reality (AR) headset to co-localize an image data set, containing a radiopaque marker, with a body of a person as viewed through the AR headset, comprising:
at least one processor of the AR headset;
a memory device of the AR headset including instructions that, when executed by the at least one processor, cause the system to:
obtaining an x-ray generated image of at least a portion of the body of the person represented in the image data set;
aligning the image data set to the x-ray generated image by using data fitting to align identified anatomical structures in the image data set and the x-ray generated image;
registering one or more additional optical codes in a coordinate system of one or more initial optical codes;
receiving a notification that one or more additional optical codes have moved; and
utilizing the additional optical codes to maintain alignment of the image data set with the body of the person.

26. The system of claim 25, wherein utilizing the additional optical codes further comprises emphasizing use of the additional optical codes to maintain alignment of the image data set with the body of the person.

27. The system of claim 25, further comprising re-aligning the image data set with the body of the person using the one or more additional optical codes that have been emphasized.

28. The system of claim 25, further comprising identifying an additional optical code added to the body of the person that is attached to at least one of: a bone of the body of the person, a bone pin in a bone of the body of the person, an organ, a blood vessel or an inner tissue of the body of the person.

29. The system of claim 25, further comprising identifying one or more additional optical codes that have become displaced optical codes due to a change in relative distance from at least one of: a visible landmark of the body of the person, a visible anatomical feature of the body of the person, a visible facial feature, a visible bone protrusion, a visible tissue protrusion, or a visible body contour.

30. The system of claim 29, wherein emphasizing use of the additional optical codes further comprises terminating use of the displaced optical codes for purposes of aligning the image data set with the body of the person.

31. The system as in claim 25, wherein the radiopaque marker is a metal marker or titanium marker.
